(19) United States
(12) Patent Application Publication
(10) Pub. No.: US 2026/0098016 A1
(43) Pub. Date: Apr. 9, 2026

(54) METHOD AND SYSTEM FOR FULLY CONTINUOUS-FLOW PREPARATION OF NITROIMIDAZOLE ANTIMICROBIAL AGENT

(71) Applicant: Fudan University, Shanghai (CN)

(72) Inventors: Fener CHEN, Shanghai (CN); Minjie LIU, Shanghai (CN); Li WAN, Shanghai (CN); Yajiao ZHANG, Shanghai (CN); Xingpeng CAI, Shanghai (CN); Zhisheng YANG, Shanghai (CN)

(21) Appl. No.: 19/417,229

(22) Filed: Dec. 11, 2025

(30) Foreign Application Priority Data
Sep. 10, 2025 (CN) 202511286756.1

(51) Int. Cl. C07D 233/92; B01J 19/00

(52) U.S. Cl. CPC C07D 233/92; B01J 19/0006; B01J 19/0093; B01J 2219/00094; B01J 2219/00101; B01J 2219/00162; B01J 2219/0086; B01J 2219/00867; B01J 2219/00873; B01J 2219/00889; B01J 2219/00963

(57) ABSTRACT

A method for fully continuous-flow preparation of a nitroimidazole antimicrobial agent using a fully continuous-flow system is provided herein. The method adopts a raw material of a glyoxal aqueous solution, an acetaldehyde aqueous solution and an ammonium hydroxide solution followed by multi-step chemical reactions and continuous post-treatments to yield a nitroimidazole antimicrobial agent with a high purity. The fully continuous-flow system for implementing the method includes a plurality of micromixers, a plurality of microreactors, a plurality of back pressure valves, a plurality of gas-liquid separators, a first plurality of online solvent switching units and a feed unit.

### CROSS-REFERENCE TO RELATED APPLICATIONS

This application claims the benefit of priority from Chinese Patent Application No. 202511286756.1, field on Sep. 10, 2025. The content of the aforementioned application, including any intervening amendments thereto, is incorporated herein by reference in its entirety.

### TECHNICAL FIELD

This application relates to pharmaceutical chemicals, and more particularly to a method and system for fully continuous-flow preparation of a nitroimidazole antimicrobial agent.

### BACKGROUND

Nitroimidazoles have been extensively used as antiprotozoal agents in the treatment of anaerobic bacterial infections and trichomoniasis, as well as the prevention of postoperative infections. The related products are predominated by metronidazole, tinidazole, ornidazole and secnidazole, whose global demands are continuously growing.

Currently, the production of nitroimidazole antimicrobial agents predominantly employs a conventional batch reactor process, which struggles with prolonged reaction time, high safety risk of nitrification, substantial sulfuric acid consumption, and high energy consumption. Additionally, the conventional process also suffers from poor conversion rate of the starting material 2-methyl-5-nitroimidazole and serious discharge of waste acids and salts. Chinese Patent Publication No. 110669011A discloses a microtubule reaction process for synthesizing metronidazole, in which 2-methyl-5-nitroimidazole and ethylene oxide are mixed in a pre-packed coiled tube reactor and then fed into another coiled tube reactor with an inner diameter of 4-20 mm to undergo a hydroxyethylation reaction to give the desired product. This approach effectively avoids the volatilization of the ethylene oxide with a low boiling point and improves its utilization efficiency. However, this method requires pre-cooling of the ethylene oxide, thereby increasing the energy expenditure. Moreover, due to the low boiling point, ethylene oxide is extremely prone to vaporization during the transport process, which will affect the metering accuracy, thereby affecting the conversion rate and selectivity of the reaction. Chinese Patent Publication No. 110922362A discloses a method of preparing ornidazole and secnidazole from 2-methyl-5-nitroimidazole, which involves the Lewis acid-mediated catalysis and consumption of organic solvents, leading to high costs and high pollution risks. Chinese Patent Publication No. 110590677A discloses a method of preparing tinidazole from 2-methyl-5-nitroimidazole, which also requires Lewis acids and organic solvents, and thus struggles with the similar defects.

### SUMMARY

To address the problems in the prior art, the present disclosure provides a method and system for fully continuous-flow preparation of nitroimidazole antimicrobial agents, having advantages of high production efficiency, excellent product quality and low discharge of three wastes.

A method for fully continuous-flow preparation of a nitroimidazole antimicrobial agent using a fully continuous-flow system, the fully continuous-flow system comprising a first micromixer, a second micromixer, a third micromixer, a fourth micromixer, a fifth micromixer, a sixth micromixer, a first microreactor, a second microreactor, a third microreactor, a fourth microreactor, a fifth microreactor, a sixth microreactor, a seventh microreactor, a first back pressure valve, a second back pressure valve, a third back pressure valve, a fourth back pressure valve, a fifth back pressure valve, a first gas-liquid separator, a second gas-liquid separator, a third gas-liquid separator, a fourth gas-liquid separator, a first online solvent switching unit, a second online solvent switching unit, a third online solvent switching unit and a feed unit; and the fully continuous-flow preparation method comprising:
(1) mixing a first glyoxal aqueous solution, a first acetaldehyde aqueous solution and a first ammonia solution in the first micromixer to obtain a first mixed solution; feeding the first mixed solution to the first microreactor followed by reaction to generate a first reaction product containing 2-methylimidazole; removing unreacted glyoxal, acetaldehyde, ammonia and water from the first reaction product in the first online solvent switching unit to collect 2-methylimidazole; and mixing 2-methylimidazole product with a sulfuric acid solution to give a mixture A;
(2) mixing the mixture A with a first nitric acid aqueous solution in the second micromixer to give a second mixed solution; transporting the second mixed solution to the second microreactor for reaction to produce a second reaction product; allowing the second reaction product to flow through the first back pressure valve and the first gas-liquid separator for gas removal to enter the third microreactor for reaction to give a third reaction product; and allowing the third reaction product to flow through the second back pressure valve and the second gas-liquid separator for gas removal to give a mixture B;
(3) mixing a second glyoxal aqueous solution, a second acetaldehyde aqueous solution and a second ammonia solution in the third micromixer to obtain a third mixed solution; feeding the third mixed solution to the fourth microreactor followed by reaction to obtain a fourth reaction product containing 2-methylimidazole; removing unreacted glyoxal, acetaldehyde, ammonia and water from the fourth reaction product in the second online solvent switching unit to collect 2-methylimidazole; and mixing 2-methylimidazole with the mixture B to give a mixture C;
(4) mixing the mixture C with a second nitric acid aqueous solution in the fourth micromixer to give a fourth mixed solution; transporting the fourth mixed solution to the fifth microreactor for reaction to produce a fifth reaction product; allowing the fifth reaction product to flow through the third back pressure valve and the third gas-liquid separator for gas removal to enter the sixth microreactor for reaction to give a sixth reaction product; and allowing the sixth reaction product to flow through the fourth back pressure valve and the fourth gas-liquid separator for gas removal to give a mixture D;
(5) mixing the mixture D with a formic acid solution in the fifth micromixer to obtain a fifth mixed solution; feeding a reagent to the sixth micromixer via the feed unit, wherein the reagent is selected from the group consisting of ethylene oxide, propylene oxide, epichlorohydrin and 2-(ethylsulfonyl)ethanol; mixing the fifth mixed solution with the reagent in the sixth micromixer to give a sixth mixed solution; feeding the sixth mixed solution to the seventh microreactor followed by reaction to obtain a seventh reaction product containing nitroimidazole; allowing the seventh reaction product to flow through the fifth back pressure valve to enter the third online solvent switching unit for removal and recovery of formic acid to give a crude nitroimidazole product; and
(6) adjusting the crude nitroimidazole product to pH 1-6 in a stirring vessel of the third online solvent switching unit followed by filtration to give a crude 2-methyl-5-nitroimidazole product; and adjusting the crude 2-methyl-5-nitroimidazole product to pH 7-13 followed by filtration, decolorization, filtration, concentration, recrystallization, filtration and drying to yield the nitroimidazole antimicrobial agent with a purity of greater than 99.9%.

In some embodiments, in steps (1)-(5), each of the first micromixer, the second micromixer, the third micromixer, the fourth micromixer, the fifth micromixer and the sixth micromixer is specially designed and manufactured according to the present disclosure, and is structurally shown in FIG. 3; each of the first micromixer, the second micromixer, the third micromixer, the fourth micromixer, the fifth micromixer and the sixth micromixer is configured as a Ω-shaped plate-type microchannel structure with an inner diameter of 200μm-30 mm and an applicable flux of 1-5,000 mL/min; the Ω-shaped plate-type microchannel structure is provided with an included angle to enhance molecular collision and mixing.

In some embodiments, in steps (1)-(5), each of the first microreactor, the second microreactor, the third microreactor, the fourth microreactor, the fifth microreactor, the sixth microreactor and the seventh microreactor is configured as an X-shaped plate-type microchannel structure with a fluid channel dimension of 200 μm-30 mm, or as a tabular-baffle filling-type microchannel structure with a fluid channel dimension of 500 μm-80 mm.

In some embodiments, in step (1) and step (3), each of the first online solvent switching unit, the second online solvent switching unit and the third online solvent switching unit is a thin-film evaporator equipped with a vacuum device, a cooling device and a thermal circulation device;
wherein the cooling device is an Allihn condenser or a Graham condenser; the vacuum device is a vacuum pump; the thermal circulation device is a heating-cooling integrated machine or a circulating oil bath; the vacuum device, the cooling device and the thermal circulation device are respectively connected to the thin-film evaporator through a first pipe and a flange;
a tank body of the thin-film evaporator is configured as a jacketed heat-exchange structure; a bottom of the thin-film evaporator is provided with a heat-exchange fluid inlet, and a top of the thin-film evaporator is provided with a heat-exchange fluid outlet; and a serpentine baffle is arranged in a heat-exchange microchannel of the thin-film evaporator and is configured to allow a heat-exchange wall to be evenly heated and avoid a heat-exchange dead area;
a material inlet is arranged on a top of the thin-film evaporator, wherein the first reaction product containing 2-methylimidazole in step (1), the fourth reaction product containing 2-methylimidazole in step (3) and the seventh reaction product containing nitroimidazole in step (5) enter the thin-film evaporator through the material inlet; a lower end of the material inlet is configured to extend to ⅔ of a height of the tank body;
the top of the thin-film evaporator is provided with a first outlet, and the first outlet is connected to a top of the cooling device through a second pipe; a bottom of the cooling device is provided with a second outlet, and the second outlet is connected to a liquid storage tank to collect condensed liquid; the liquid storage tank is sequentially connected to a vacuum regulating valve and the vacuum device through a third pipe, wherein the vacuum pump is configured to provide a negative pressure, and the vacuum regulating valve is configured to adjust a negative pressure in the stirring vessel and the cooling device, so as to control a vaporization rate of low-boiling compounds and ensure complete condensation;
a heat-exchange fluid is pumped into the thin-film evaporator through a circulation pump; and
after removing low-boiling compounds and simultaneously adding a sulfuric acid solution, a solvent of the reaction product is switched to the sulfuric acid solution; two thin-film evaporators connected to each other in parallel are adopted herein to enable a continuous-flow operation such as a continuous-flow preparation of the mixture A and the mixture C, and a continuous-flow application of a next reaction.

In some embodiments, in step (5), the feed unit is a buffer tank; an inlet pipe of the reagent is arranged at ⅓ of a height of the buffer tank; an outlet pipe of the reagent is configured to extend to a position at a distance of ⅛- 1/10 of a height of the buffer tank from a bottom of the buffer tank; the buffer tank is pressurized with nitrogen to 3-15 bar after filled with the reagent, so as to prevent the reagent from vaporizing at room temperature, thereby ensuring stable feeding; and the reagent is quantitively pumped by a plunger pump to be sequentially transported to the sixth micromixer for mixing with a fifth mixed solution.

In some embodiments, in step (6), the crude nitroimidazole product is adjusted to pH 1-6 with an alkaline aqueous solution, monitored by an online pH meter and transported by a first peristaltic pump to a first online filter for filtration to collect a filtrate as the crude 2-methyl-5-nitroimidazole product; the filtrate is adjusted to pH 7-13 with ammonia solution or liquid ammonia in a pH adjustment vessel, and transported by a second peristaltic pump to a second online filter for filtration, and a filter cake is collected by a rotary scraper, and subjected to decolorization, filtration, concentration, recrystallization, filtration and drying to yield the nitroimidazole antimicrobial agent with a purity of greater than 99.9%.

In some embodiments, a molar ratio of the glyoxal to the acetaldehyde to the ammonia solution to the sulfuric acid to the formic acid to the reagent is 1:(1.0-2.0):(2.0-4.0):(1.0-5.0):(1.0-5.0):(1.0-5.0); and preferably, 1:(1.0-1.5):(2.0-3.0):(1.0-3.0):(1.0-3.0):(1.0-3.0).

In some embodiments, a system for implementing the aforementioned method, comprising the first micromixer, the second micromixer, the third micromixer, the fourth micromixer, the fifth micromixer, the sixth micromixer, the first microreactor, the second microreactor, the third microreactor, the fourth microreactor, the fifth microreactor, the sixth microreactor, the seventh microreactor, the first back pressure valve, the second back pressure valve, the third back pressure valve, the fourth back pressure valve, the fifth back pressure valve, the first gas-liquid separator, the second gas-liquid separator, the third gas-liquid separator, the fourth gas-liquid separator, the first online solvent switching unit, the second online solvent switching unit, the third online solvent switching unit and the feed unit.

Compared to the prior art, the present disclosure has the following beneficial effects.

The present disclosure provides a reasonable arrangement of the micromixers, the microreactors and the online solvent switching units based on the method for fully continuous-flow preparation of a nitroimidazole antimicrobial agent, so as to enable a yield of greater than 90% and a purity of greater than 99.9%. The aforementioned method and system pioneeringly enable a recycle of the nitration solution, significantly reduce a consumption of sulfuric acid by more than 60%, and also enable a recycle of formic acid. As a consequence, the aforementioned method and system effectively minimizes a production of waste acid and waste salt, so as to achieve a significant cost reduction and an efficiency enhancement. Furthermore, a self-designed delivery system of the reagent with precise temperature and pressure control ensures an accurate and stable supply of the reagent to a pressurized fully continuous-flow microreactor.

### BRIEF DESCRIPTION OF THE DRAWINGS

FIG. 1 is a flow chart of a method for fully continuous-flow preparation of a nitroimidazole antimicrobial agent according to an embodiment of the present disclosure;

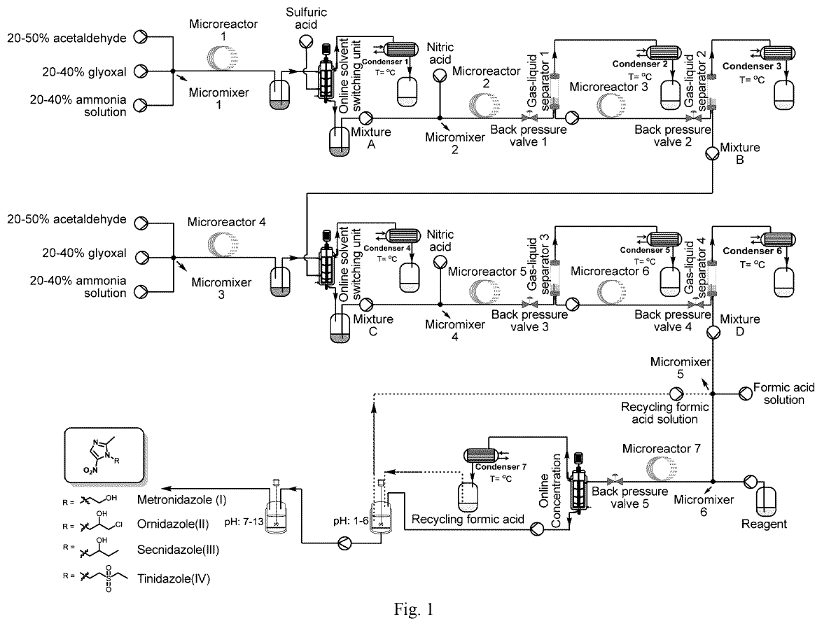

FIG. 2 structurally shows a gas-liquid separator according to an embodiment of the present disclosure;

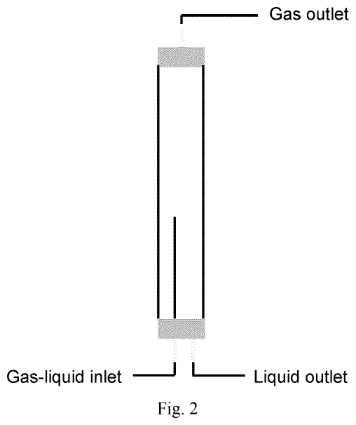

FIG. 3 structurally shows a Ω-shaped micromixer according to an embodiment of the present disclosure; and

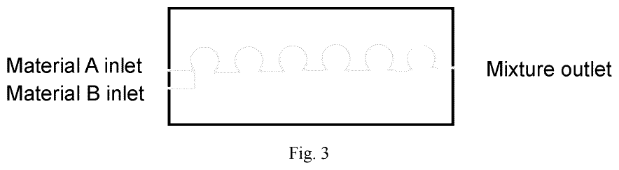

FIG. 4 structurally shows a feed unit according to an embodiment of the present disclosure.

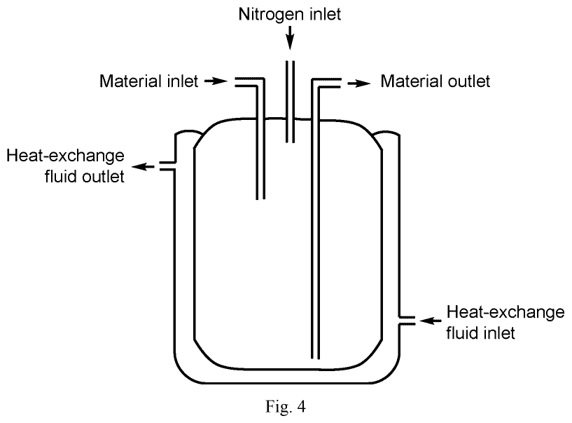

### DETAILED DESCRIPTION OF EMBODIMENTS

In order to introduce the technical solutions, configuration features, objects and beneficial effects of the present disclosure detailly, the present disclosure will be further described in combination with the embodiments and the accompanying figures. The embodiments are implemented on the premise of the technical solutions of the present disclosure, and provide detail implement methods and specific operational processes. The embodiments disclosed herein are not intended to limit the present disclosure.

To better introduce the objects, technical solutions and advantages of the present disclosure, the present disclosure will be further described in combination with the embodiments.

### Example 1

A first glyoxal aqueous solution and a first acetaldehyde aqueous solution were respectively pumped by a plunger pump based on a molar ratio of 1:1 to be sequentially mixed via a three-way valve and transported to a first micromixer. A first ammonia solution (2.0 eq) was pumped by a plunger pump to be sequentially mixed with the first glyoxal aqueous solution and the first acetaldehyde aqueous solution in the first micromixer, so as to obtain a first mixed solution. The first mixed solution was fed to a first microreactor followed by reaction to generate a first reaction product containing 2-methylimidazole. Unreacted glyoxal, acetaldehyde ammonia and water were removed from the first reaction product in a first online solvent switching unit to collect 2-methylimidazole. The 2-methylimidazole was mixed with a sulfuric acid solution (1.5 eq) to give a mixture A.

The mixture A was mixed with a first nitric acid aqueous solution (1.2 eq) in a second micromixer to give a second mixed solution. The second mixed solution was transported to the second microreactor for reaction to produce a second reaction product. The second reaction product was allowed to flow through a first back pressure valve and a first gas-liquid separator for gas removal to enter a third microreactor for reaction to give a third reaction product. The third reaction product was allowed to flow through a second back pressure valve and a second gas-liquid separator for gas removal to give a mixture B.

A second glyoxal aqueous solution and a second acetaldehyde aqueous solution were respectively pumped by a plunger pump based on a molar ratio of 1:1 to be sequentially mixed via a three-way valve and transported to a third micromixer. A second ammonia solution (1.2 eq) was pumped by a plunger pump to be sequentially mixed with the second glyoxal aqueous solution and the second acetaldehyde aqueous solution in the third micromixer, so as to obtain a third mixed solution. The third mixed solution was fed to a fourth microreactor followed by reaction to obtain a fourth reaction product containing 2-methylimidazole. Unreacted glyoxal, acetaldehyde, ammonia and water from the fourth reaction product in a second online solvent switching unit to collect 2-methylimidazole. The 2-methylimidazole was mixed with the mixture B to give a mixture C.

The mixture C was mixed with a second nitric acid aqueous solution (1.2 eq) in a fourth micromixer to give a fourth mixed solution. The fourth mixed solution was transported to a fifth microreactor for reaction to produce a fifth reaction product. The fifth reaction product was allowed to flow through a third back pressure valve and a third gas-liquid separator for gas removal to enter a sixth microreactor for reaction to give a sixth reaction product. The sixth reaction product was allowed to flow through a fourth back pressure valve and a fourth gas-liquid separator for gas removal to give a mixture D.

The mixture D was mixed with a formic acid solution (a content of 2-methyl-5-nitroimidazole was 20%) in a fifth micromixer to obtain a fifth mixed solution. Ethylene oxide (1.2 eq) was fed to a sixth micromixer via the feed unit. The fifth mixed solution was mixed with the ethylene oxide (1.2 eq) in the sixth micromixer to give a sixth mixed solution. The sixth mixed solution was fed to a seventh microreactor followed by a ring-opening reaction to obtain a seventh reaction product containing metronidazole. The seventh reaction product was allowed to flow through a fifth back pressure valve to enter a third online solvent switching unit for removal and condensation recovery of formic acid to give a crude metronidazole product.

The crude metronidazole product was adjusted to pH 4-5 in a stirring vessel of the third online solvent switching unit followed by filtration to give a crude 2-methyl-5-nitroimidazole product. The crude 2-methyl-5-nitroimidazole product was dissolved with a recovered formic acid and adjusted to pH 11-12 followed by filtration, decolorization, filtration, concentration, recrystallization, filtration and drying to yield metronidazole with a purity of greater than 99.9%. The sulfuric acid consumption was reduced by 60% compared to a single-pass consumption. The recovery rate of formic acid reached 98%. The single-pass yield of metronidazole was 77%, while the yield of metronidazole reached 95% after recycling 2-methyl-5-nitroimidazole.

### Example 2

A first glyoxal aqueous solution and a first acetaldehyde aqueous solution were respectively pumped by a plunger pump based on a molar ratio of 1:1 to be sequentially mixed via a three-way valve and transported to the first micromixer. A first ammonia solution (2.0 eq) was pumped by a plunger pump to be sequentially mixed with the first glyoxal aqueous solution and the first acetaldehyde aqueous solution in the first micromixer to obtain a first mixed solution. The first mixed solution was fed to the first microreactor to generate a first reaction product containing 2-methylimidazole. Unreacted glyoxal, acetaldehyde, ammonia and water were removed from the first reaction in the first online solvent switching unit to collect 2-methylimidazole. The 2-methylimidazole was mixed with a sulfuric acid solution (1.8 eq) to give a mixture A.

The mixture A was mixed with a first nitric acid aqueous solution (1.2 eq) in the second micromixer to give a second mixed solution. The second mixed solution was transported to the second microreactor for reaction to produce a second reaction product. The second reaction product was allowed to flow through the first back pressure valve and the first gas-liquid separator for gas removal to enter the third microreactor for reaction to give a third reaction product. The third reaction product was allowed to flow through the second back pressure valve and the second gas-liquid separator for gas removal to give a mixture B.

A second glyoxal aqueous solution and a second acetaldehyde aqueous solution were respectively pumped by a plunger pump based on a molar ratio of 1:1 to be sequentially mixed via a three-way valve and transported to the third micromixer. A second ammonia solution (1.5 eq) was pumped by a plunger pump to be sequentially mixed with the second glyoxal aqueous solution and the second acetaldehyde aqueous solution in the third micromixer, so as to obtain a third mixed solution. The third mixed solution was fed to the fourth microreactor followed by reaction to obtain a fourth reaction product containing 2-methylimidazole. Unreacted glyoxal, acetaldehyde, ammonia and water from the fourth reaction product in the second online solvent switching unit to collect 2-methylimidazole. The 2-methylimidazole was mixed with the mixture B to give a mixture C.

The mixture C was mixed with a second nitric acid aqueous solution (1.5 eq) in the fourth micromixer to give a fourth mixed solution. The fourth mixed solution was transported to the fifth microreactor for reaction to produce a fifth reaction product. The fifth reaction product was allowed to flow through the third back pressure valve and the third gas-liquid separator for gas removal to enter the sixth microreactor for reaction to give a sixth reaction product. The sixth reaction product was allowed to flow through the fourth back pressure valve and the fourth gas-liquid separator for gas removal to give a mixture D.

The mixture D was mixed with a formic acid solution (a content of 2-methyl-5-nitroimidazole was 20%) in the fifth micromixer to obtain a fifth mixed solution. Propylene oxide (1.2 eq) was fed to the sixth micromixer via the feed unit. The fifth mixed solution was mixed with the propylene oxide (1.2 eq) in the sixth micromixer to give a sixth mixed solution. The sixth mixed solution was fed to the seventh microreactor followed by a ring-opening reaction to obtain a seventh reaction product containing secnidazole. The seventh reaction product was allowed to flow through the fifth back pressure valve to the third online solvent switching unit followed by removal and condensation recovery of formic acid to give a crude secnidazole product.

The crude secnidazole product was adjusted to pH 4-5 in a stirring vessel of the third online solvent switching unit followed by filtration to give a crude 2-methyl-5-nitroimidazole product. The crude 2-methyl-5-nitroimidazole product was dissolved with a recovered formic acid and adjusted to pH 11-12 followed by filtration, decolorization, filtration, concentration, recrystallization, filtration and drying to yield secnidazole with a purity of greater than 99.9%. The sulfuric acid consumption was reduced by 61% compared to a single-pass consumption. The recovery rate of formic acid reached 99%. The single-pass yield of secnidazole was 82%, while the yield of secnidazole reached 95% after recycling 2-methyl-5-nitroimidazole.

### Example 3

A first glyoxal aqueous solution and a first acetaldehyde aqueous solution were respectively pumped by a plunger pump based on a molar ratio of 1:1 to be sequentially mixed via a three-way valve and transported to the first micromixer. A first ammonia solution (2.0 eq) was pumped by a plunger pump to be sequentially mixed with the first glyoxal aqueous solution and the first acetaldehyde aqueous solution in the first micromixer, so as to obtain a first mixed solution. The first mixed solution was fed to the first microreactor followed by reaction to generate a first reaction product containing 2-methylimidazole. Unreacted glyoxal, acetaldehyde, ammonia and water were removed from the first reaction product in the first online solvent switching unit to collect 2-methylimidazole. The 2-methylimidazole was mixed with a sulfuric acid (1.8 eq) to give a mixture A.

The mixture A was mixed with a first nitric acid aqueous solution (1.2 eq) in the second micromixer to give a second mixed solution. The second mixed solution was transported to the second microreactor for reaction to produce a second reaction product. The second reaction product was allowed to flow through the first back pressure valve and the first gas-liquid separator for gas removal to enter the third microreactor for reaction to give a third reaction product. The third reaction product was allowed to flow through the second back pressure valve and the second gas-liquid separator for gas removal to give a mixture B.

A second glyoxal aqueous solution and a second acetaldehyde aqueous solution were respectively pumped by a plunger pump based on a molar ratio of 1:1 to be sequentially mixed via a three-way valve and transported to the third micromixer. A second ammonia solution (1.5 eq) was pumped by a plunger pump to be sequentially mixed with the second glyoxal aqueous solution and the second acetaldehyde aqueous solution in the third micromixer, so as to obtain a third mixed solution. The third mixed solution was fed to the fourth microreactor followed by reaction to obtain a fourth reaction product containing 2-methylimidazole. Unreacted glyoxal, acetaldehyde, ammonia and water from the fourth reaction product in the second online solvent switching unit to collect 2-methylimidazole. The 2-methylimidazole was mixed with the mixture B to give a mixture C.

The mixture C was mixed with a second nitric acid aqueous solution (1.5 eq) in the fourth micromixer to give a fourth mixed solution. The fourth mixed solution was transported to the fifth microreactor for reaction to produce a fifth reaction product. The fifth reaction product was allowed to flow through the third back pressure valve and the third gas-liquid separator for gas removal to enter the sixth microreactor for reaction to give a sixth reaction product. The sixth reaction product was allowed to flow through the fourth back pressure valve and the fourth gas-liquid separator for gas removal to give a mixture D.

The mixture D was mixed with a formic acid solution (a content of 2-methyl-5-nitroimidazole was 20%) in the fifth micromixer to obtain a fifth mixed solution. Epichlorohydrin (1.2 eq) was fed to the sixth micromixer via the feed unit. The fifth mixed solution was mixed with the epichlorohydrin (1.2 eq) in the sixth micromixer to give a sixth mixed solution. The sixth mixed solution was fed to the seventh microreactor followed by a ring-opening reaction to obtain a seventh reaction product containing ornidazole. The seventh reaction product was allowed to flow through the fifth back pressure valve to enter the third online solvent switching unit for removal and condensation recovery of formic acid to give a crude ornidazole product.

The crude ornidazole product was adjusted to pH 4-5 in a stirring vessel of the third online solvent switching unit followed by filtration to give a crude 2-methyl-5-nitroimidazole product. The crude 2-methyl-5-nitroimidazole product was dissolved with a recovered formic acid and adjusted to pH 8-9 followed by filtration, decolorization, filtration, concentration, recrystallization, filtration and drying to yield ornidazole with a purity of greater than 99.9%. The sulfuric acid consumption was reduced by 62% compared to a single-pass consumption. The recovery rate of formic acid reached 98%. The single-pass yield of ornidazole was 85%, while the yield of ornidazole reached 94% after recycling 2-methyl-5-nitroimidazole.

### Example 4

A first glyoxal aqueous solution and a first acetaldehyde aqueous solution were respectively pumped by a plunger pump based on a molar ratio of 1:1 to be sequentially mixed via a three-way valve and transported to the first micromixer. A first ammonia solution (2.0 eq) was pumped by a plunger pump to be sequentially mixed with the first glyoxal aqueous solution and the first acetaldehyde aqueous solution in the first micromixer, so as to obtain a first mixed solution. The first mixed solution was fed to the first microreactor followed by reaction to generate a first reaction product containing 2-methylimidazole. Unreacted glyoxal, acetaldehyde, ammonia and water were removed from the first reaction product in the first online solvent switching unit to collect 2-methylimidazole. The 2-methylimidazole was mixed with a sulfuric acid solution (1.8 eq) to give a mixture A.

The mixture A was mixed with a first nitric acid aqueous solution (1.2 eq) in the second micromixer to give a second mixed solution. The second mixed solution was transported to the second microreactor for reaction to produce a second reaction product. The second reaction product was allowed to flow through the first back pressure valve and the first gas-liquid separator for gas removal to enter the third microreactor for reaction to give a third reaction product. The third reaction product was allowed to flow through the second back pressure valve and the second gas-liquid separator for gas removal to give a mixture B.

A second glyoxal aqueous solution and a second acetaldehyde aqueous solution were respectively pumped by a plunger pump based on a molar ratio of 1:1 to be sequentially mixed via a three-way valve and transported to the third micromixer. A second ammonia solution (1.5 eq) was pumped by a plunger pump to be sequentially mixed with the second glyoxal aqueous solution and the second acetaldehyde aqueous solution in the third micromixer, so as to obtain a third mixed solution. The third mixed solution was fed to the fourth microreactor followed by reaction to obtain a fourth reaction product containing 2-methylimidazole. Unreacted glyoxal, acetaldehyde, ammonia and water from the fourth reaction product in the second online solvent switching unit to collect 2-methylimidazole. The 2-methylimidazole was mixed with the mixture B to give a mixture C.

The mixture C was mixed with a second nitric acid aqueous solution (1.5 eq) in the fourth micromixer to give a fourth mixed solution. The fourth mixed solution was transported to the fifth microreactor for reaction to produce a fifth reaction product. The fifth reaction product was allowed to flow through the third back pressure valve and the third gas-liquid separator for gas removal to enter the sixth microreactor for reaction to give a sixth reaction product. The sixth reaction product was allowed to flow through the fourth back pressure valve and the fourth gas-liquid separator for gas removal to give a mixture D.

The mixture D was mixed with a formic acid solution (a content of 2-methyl-5-nitroimidazole was 20%) in the fifth micromixer to obtain a fifth mixed solution. 2-(ethylsulfonyl)ethanol (1.2 eq) was fed to the sixth micromixer via the feed unit. The fifth mixed solution was mixed with the 2-(ethylsulfonyl)ethanol (1.2 eq) in the sixth micromixer to give a sixth mixed solution. The sixth mixed solution was fed to the seventh microreactor followed by a ring-opening reaction to obtain a seventh reaction product containing tinidazole. The seventh reaction product was allowed to flow through the fifth back pressure valve to enter the third online solvent switching unit for removal and condensation recovery of formic acid to give a crude tinidazole product.

The crude tinidazole product was adjusted to pH 4-5 in a stirring vessel of the third online solvent switching unit followed by filtration to give a crude 2-methyl-5-nitroimidazole product. The crude 2-methyl-5-nitroimidazole product was dissolved with a recovered formic acid and adjusted to pH 11-12 followed by filtration, decolorization, filtration, concentration, recrystallization, filtration and drying to yield tinidazole with a purity of greater than 99.9%. The sulfuric acid consumption was reduced by 60% compared to a single-pass consumption. The recovery rate of formic acid reached 98%. The single-pass yield of tinidazole was 78%, while the yield of tinidazole reached 93% after recycling 2-methyl-5-nitroimidazole.

What is claimed is:

1. A method for fully continuous-flow preparation of a nitroimidazole antimicrobial agent using a fully continuous-flow system, the fully continuous-flow system comprising a first micromixer, a second micromixer, a third micromixer, a fourth micromixer, a fifth micromixer, a sixth micromixer, a first microreactor, a second microreactor, a third microreactor, a fourth microreactor, a fifth microreactor, a sixth microreactor, a seventh microreactor, a first back pressure valve, a second back pressure valve, a third back pressure valve, a fourth back pressure valve, a fifth back pressure valve, a first gas-liquid separator, a second gas-liquid separator, a third gas-liquid separator, a fourth gas-liquid separator, a first online solvent switching unit, a second online solvent switching unit, a third online solvent switching unit and a feed unit, and the method comprising:
(1) mixing a first glyoxal aqueous solution, a first acetaldehyde aqueous solution and a first ammonia solution in the first micromixer to obtain a first mixed solution; feeding the first mixed solution to the first microreactor followed by reaction to generate a first reaction product containing 2-methylimidazole; removing unreacted glyoxal, acetaldehyde, ammonia and water from the first reaction product in the first online solvent switching unit to collect 2-methylimidazole; and mixing 2-methylimidazole with a sulfuric acid solution to give a mixture A;
(2) mixing the mixture A with a first nitric acid aqueous solution in the second micromixer to give a second mixed solution; transporting the second mixed solution to the second microreactor for reaction to produce a second reaction product; allowing the second reaction product to flow through the first back pressure valve and the first gas-liquid separator for gas removal to enter the third microreactor for reaction to give a third reaction product; and allowing the third reaction product to flow through the second back pressure valve and the second gas-liquid separator for gas removal to give a mixture B;
(3) mixing a second glyoxal aqueous solution, a second acetaldehyde aqueous solution and a second ammonia solution in the third micromixer to obtain a third mixed solution; feeding the third mixed solution to the fourth microreactor followed by reaction to obtain a fourth reaction product containing 2-methylimidazole; removing unreacted glyoxal, acetaldehyde, ammonia and water from the fourth reaction product in the second online solvent switching unit to collect 2-methylimidazole; and mixing 2-methylimidazole with the mixture B to give a mixture C;
(4) mixing the mixture C with a second nitric acid aqueous solution in the fourth micromixer to give a fourth mixed solution; transporting the fourth mixed solution to the fifth microreactor for reaction to produce a fifth reaction product; allowing the fifth reaction product to flow through the third back pressure valve and the third gas-liquid separator for gas removal to enter the sixth microreactor for reaction to give a sixth reaction product; and allowing the sixth reaction product to flow through the fourth back pressure valve and the fourth gas-liquid separator for gas removal to give a mixture D;
(5) mixing the mixture D with a formic acid solution in the fifth micromixer to obtain a fifth mixed solution; feeding a reagent to the sixth micromixer via the feed unit, wherein the reagent is selected from the group consisting of ethylene oxide, propylene oxide, epichlorohydrin and 2-(ethylsulfonyl)ethanol; mixing the fifth mixed solution with the reagent in the sixth micromixer to give a sixth mixed solution; feeding the sixth mixed solution to the seventh microreactor followed by reaction to obtain a seventh reaction product containing nitroimidazole; allowing the seventh reaction product to flow through the fifth back pressure valve to enter the third online solvent switching unit for removal and recovery of formic acid to give a crude nitroimidazole product; and
(6) adjusting the crude nitroimidazole product to pH 1-6 in a stirring vessel of the third online solvent switching unit followed by filtration to give a crude 2-methyl-5-nitroimidazole product; and adjusting the crude 2-methyl-5-nitroimidazole product to pH 7-13 followed by filtration, decolorization, filtration, concentration, recrystallization, filtration and drying to yield the nitroimidazole antimicrobial agent with a purity of greater than 99.9%.

2. The method of claim 1, wherein each of the first micromixer, the second micromixer, the third micromixer, the fourth micromixer, the fifth micromixer and the sixth micromixer is configured as a Ω-shaped plate-type microchannel structure with an inner diameter of 200 μm-30 mm and an applicable flux of 1-5,000 mL/min, so as to enhance molecular collision and mixing.

3. The method of claim 1, wherein each of the first microreactor, the second microreactor, the third microreactor, the fourth microreactor, the fifth microreactor, the sixth microreactor and the seventh microreactor is configured as an X-shaped plate-type microchannel structure with a fluid channel dimension of 200 μm-30 mm, or as a tubular-baffle filling-type microchannel structure with a fluid channel dimension of 500 μm-80 mm.

4. The method of claim 1, wherein each of the first online solvent switching unit, the second online solvent switching unit and the third online solvent switching unit is a thin-film evaporator equipped with a vacuum device, a cooling device and a thermal circulation device;
the cooling device is an Allihn condenser or a Graham condenser; the vacuum device is a vacuum pump; the thermal circulation device is a heating-cooling integrated machine or a circulating oil bath; the vacuum device, the cooling device and the thermal circulation device are respectively connected to the thin-film evaporator through a first pipe and a flange;
a tank body of the thin-film evaporator is configured as a jacketed heat-exchange structure; a bottom of the thin-film evaporator is provided with a heat-exchange fluid inlet, and a top of the thin-film evaporator is provided with a heat-exchange fluid outlet; and a serpentine baffle is arranged in a heat-exchange microchannel of the thin-film evaporator;
a material inlet is arranged on a top of the thin-film evaporator, wherein the first reaction product containing 2-methylimidazole in step (1), the fourth reaction product containing 2-methylimidazole in step (3) and the seventh reaction product containing nitroimidazole in step (5) enter the thin-film evaporator through the material inlet; a lower end of the material inlet is configured to extend to ⅔ of a height of the tank body;
the top of the thin-film evaporator is provided with a first outlet, and the first outlet is connected to a top of the cooling device through a second pipe; a bottom of the cooling device is provided with a second outlet, and the second outlet is connected to a liquid storage tank to collect condensed liquid; the liquid storage tank is sequentially connected to a vacuum regulating valve and the vacuum device through a third pipe, wherein the vacuum pump is configured to provide a negative pressure, and the vacuum regulating valve is configured to adjust a negative pressure in the stirring vessel and the cooling device; and
a heat-exchange fluid is pumped into the thin-film evaporator through a circulation pump.

5. The method of claim 1, wherein in step (5), the feed unit is a buffer tank; an inlet pipe of the reagent is arranged at ⅓ of a height of the buffer tank; an outlet pipe of the reagent is configured to extend to a position at a distance of ⅛- 1/10 of a height of the buffer tank from a bottom of the buffer tank; the buffer tank is pressurized with nitrogen to 3-15 bar after filled with the reagent; and the reagent is quantitively pumped by a plunger pump to be sequentially transported to the sixth micromixer for mixing with a fifth mixed solution.

6. The method of claim 1, wherein in step (6), the crude nitroimidazole product is adjusted to pH 1-6 with an alkaline aqueous solution, monitored by an online pH meter and transported by a first peristaltic pump to a first online filter for filtration to collect a filtrate as the crude 2-methyl-5-nitroimidazole product; the filtrate is adjusted to pH 7-13 with ammonia solution or liquid ammonia in a pH adjustment vessel, and transported by a second peristaltic pump to a second online filter for filtration, and a filter cake is collected by a rotary scraper, and subjected to decolorization, filtration, concentration, recrystallization, filtration and drying to yield the nitroimidazole antimicrobial agent with a purity of greater than 99.9%.

7. A fully system for implementing the method of claim 1, comprising the first micromixer, the second micromixer, the third micromixer, the fourth micromixer, the fifth micromixer, the sixth micromixer, the first microreactor, the second microreactor, the third microreactor, the fourth microreactor, the fifth microreactor, the sixth microreactor, the seventh microreactor, the first back pressure valve, the second back pressure valve, the third back pressure valve, the fourth back pressure valve, the fifth back pressure valve, the first gas-liquid separator, the second gas-liquid separator, the third gas-liquid separator, the fourth gas-liquid separator, the first online solvent switching unit, the second online solvent switching unit, the third online solvent switching unit and the feed unit;
wherein each of the first micromixer, the second micromixer, the third micromixer, the fourth micromixer, the fifth micromixer and the sixth micromixer is configured as a Ω-shaped plate-type microchannel structure with an inner diameter of 200 μm-30 mm and an applicable flux of 1-5,000 mL/min, so as to enhance molecular collision and mixing;
each of the first microreactor, the second microreactor, the third microreactor, the fourth microreactor, the fifth microreactor, the sixth microreactor and the seventh microreactor is configured as an X-shaped plate-type microchannel structure with a fluid channel dimension of 200 μm-30 mm, or as a tabular-baffle filling-type microchannel structure with a fluid channel dimension of 500 μm-80 mm;
each of the first online solvent switching unit, the second online solvent switching unit and the third online solvent switching unit is a thin-film evaporator, which is equipped with a vacuum device, a cooling device and a thermal circulation device;
the cooling device is an Allihn condenser or a Graham condenser; the vacuum device is a vacuum pump; the thermal circulation device is a heating-cooling integrated machine or a circulating oil bath; the vacuum device, the cooling device and the thermal circulation device are respectively connected to the thin-film evaporator through a first pipe and a flange;
a tank body of the thin-film evaporator is configured as a jacketed heat-exchange structure; a bottom of the thin-film evaporator is provided with a heat-exchange fluid inlet, and a top of the thin-film evaporator is provided with a heat-exchange fluid outlet; and a serpentine baffle is arranged in a heat-exchange microchannel of the thin-film evaporator;
a material inlet is arranged on a top of the thin-film evaporator, wherein the first reaction product containing 2-methylimidazole in step (1), the fourth reaction product containing 2-methylimidazole in step (3) and the seventh reaction product containing nitroimidazole in step (5) enter the thin-film evaporator through the material inlet; a lower end of the material inlet is configured to extend to ⅔ of a height of the tank body;
the top of the thin-film evaporator is provided with a first outlet, and the first outlet is connected to a top of the cooling device through a second pipe; a bottom of the cooling device is provided with a second outlet, and the second outlet is connected to a liquid storage tank to collect condensed liquid; the liquid storage tank is sequentially connected to a vacuum regulating valve and the vacuum device through a third pipe, wherein the vacuum pump is configured to provide a negative pressure, and the vacuum regulating valve is configured to adjust a negative pressure in the stirring vessel and the cooling device;
a heat-exchange fluid is pumped into the thin-film evaporator through a circulation pump; and
the feed unit is a buffer tank; an inlet pipe of the reagent is arranged at ⅓ of a height of the buffer tank; an outlet pipe of the reagent is configured to extend to a position at a distance of ⅛- 1/10 of a height of the buffer tank from a bottom of the buffer tank; the buffer tank is pressurized with nitrogen to 3-15 bar after filled with the reagent; and the reagent is quantitively pumped by a plunger pump to be sequentially transported to the sixth micromixer for mixing with a fifth mixed solution.
